(19) United States
(12) Patent Application Publication
(10) Pub. No.: US 2014/0180419 A1
(43) Pub. Date: Jun. 26, 2014

(54) CONTINUOUSLY HEIGHT-ADJUSTABLE INTERVERTEBRAL FUSION IMPLANT

(71) Applicant: FACET-LINK INC., Rockaway, NJ (US)

(72) Inventor: Klaus DMUSCHEWSKY, Hamburg (DE)

(73) Assignee: FACET-LINK INC., Rockaway, NJ (US)

(21) Appl. No.: 14/107,672

(22) Filed: Dec. 16, 2013

Related U.S. Application Data
(60) Provisional application No. 61/738,267, filed on Dec. 17, 2012.

(30) Foreign Application Priority Data
Dec. 14, 2012 (DE) 20 2012 011 959.1

(51) Int. Cl. A61F 2/44

(52) U.S. Cl. CPC A61F 2/4455
USPC 623/17.16

(57) ABSTRACT

The invention relates to an intervertebral fusion implant for fusing two adjacent vertebrae, comprising a base piece and a cover piece and also a spreading means, wherein the base piece and the cover piece are connected by a rocker which is situated therebetween and externally coupled, and the spreading means is a spreader clip on the rocker and a drive device is provided for the spreader clip. The spreader clip is used to erect the rocker such that the implant expands in terms of height. As a result of the external coupling of the rocker, a large lifting travel is available. Furthermore, the spreader clip has a blocking effect in relation to an unwanted return as a result of the frictional forces thereof.

### REFERENCE TO RELATED APPLICATIONS

This application is a nonprovisional of U.S. Provisional Application No. 61/738,267, filed Dec. 17, 2012, which claims priority to German Application No. 20 2012 011 959.1, filed Dec. 14, 2012, the entire contents of which are incorporated herein by reference.

### FIELD OF THE INVENTION

The invention relates to an implant for fusing two adjacent vertebrae, comprising a base piece and a cover piece and also a spreading means.

### BACKGROUND OF THE INVENTION

The intervertebral disks of the vertebral column suffer degeneration as a result of wear or of pathological changes. If conservative treatment by medication and/or physiotherapy is ineffective, surgical treatment is sometimes indicated. In this connection, it is known for a movable or immovable implant to be inserted into the intervertebral space containing the degenerated intervertebral disk. This implant takes over the support function of the degenerated intervertebral disk and to this extent restores a stable support between the adjacent vertebrae. Immovable implants are also referred to as “fusion implants”.

Various surgical techniques are known for implanting the fusion implants. A traditional surgical technique involves a ventral access route, in order thereby to avoid the danger of damaging the spinal cord in the vertebral column. However, this advantage is obtained at the price of a very long access route through the abdominal cavity or thoracic cavity of the patient. Since this can cause complications, an alternative access route has become established, namely from the dorsal direction. Although the latter affords the advantage of a short route, there is the danger of collision with or damage to the spinal cord. To minimize this danger, the operation is usually performed by minimally invasive surgery. Approaches of this kind directly from the dorsal direction or more from the side are known as PLIF (posterior lumbar intervertebral fusion) or TLIF (transforaminal lumbar interbody fusion), in which the intervertebral disk is exposed from the posterior or lateral direction, respectively. Because of the small transverse incisions used in the approach by minimally invasive surgery, the size of the fusion implants is of course greatly restricted here.

For treatment using the PLIF or TLIF technique, very small fusion implants are known. They afford the advantage of being able to be implanted by minimally invasive surgery thanks to their small size. However, an inherent disadvantage of their small size is that the support function is limited because of the small dimensions and is sometimes inadequate. Although a larger size of the fusion implants would improve the support function, this is impractical because of the limits of minimally invasive surgery.

### SUMMARY OF THE INVENTION

The invention has set out to improve a fusion implant of the type mentioned at the outset to the extent that, while still having a small access cross section, as is conventional for minimally invasive surgery, it can nevertheless achieve an improved support effect, even in the case of large intervertebral spaces.

A solution according to the invention lies in the features broadly described herein.

In an intervertebral fusion implant for fusing two adjacent vertebrae, comprising a base piece and a cover piece and also a spreading means, provision, according to the invention, is made for the base piece and the cover piece to be connected by a rocker which is situated therebetween and externally coupled, wherein the spreading means is a spreader clip on the rocker and a drive means is provided for the spreader clip.

The invention is based on the concept of erecting the externally coupled rocker by means of the drive means, with the spreader clip acting as a transmission member in this case. By erecting the rocker, the cover piece and the base piece move apart such that the intervertebral fusion implant expands in terms of height. As a result of the external coupling of the rocker, a relatively large lifting travel is available thereby. In addition to the relatively large lifting travel, the transmission by means of the spreader clip onto the rocker affords the advantage of more points of wear, which bring about a blocking effect, being available by using the spreader clip as transmission member. This counteracts the risk of inadvertent return, i.e. that the height reduces.

The intervertebral fusion implant configured according to the invention furthermore offers the advantage of being continuously adjustable. It can therefore be adapted particularly finely to the respective anatomical conditions of the patient. A further advantage consists of the fact that, in principle, it can be returned. That is to say, if the drive means is actuated in the opposite direction, the expansion is retracted and the height of the implant is reduced. Occasionally, this is a significant advantage, particularly for testing purposes, but also for implantation at anatomically difficult sites.

Provision is expediently made for two spreader clips situated opposite to one another. This affords the advantage of a larger and a symmetrical application of force from both sides. As a result of applying force from both sides, the risk of tilting between base piece and cover piece during the lifting movement is counteracted.

An expedient embodiment of the drive means is a contraction element. In this case, the spreader clip is pulled against a counterbearing, which can also be formed by a second spreader clip, and spreads open in the process. This enables a linear embodiment, which is particularly expedient in mechanical terms and which has significant advantages in respect of the compactness thereof in view of installation space aspects. A particularly expedient embodiment lies in the use of a screwing spindle as actuator for the drive means. It enables a high force transmission while at the same, time having low installation space requirements. Furthermore, the screwing spindle affords the advantage of an inherent self-retention, as a result of which there cannot be an inadvertent return of the achieved expansion. However, on the other hand, the screwing spindle allows the retraction of the expansion in a planned manner by actuation in the opposite direction.

The rocker is preferably configured in an integral embodiment with the hinges thereof. This reduces the number of parts and simplifies the production. The integral production also ensures that use is always made of parts that fit to one another and so, unlike in the case of separate parts, there cannot be maladjustments due to inexpedient tolerances. In this case it is particularly expedient if the rocker has an integral design with the base piece and/or cover piece.

An embodiment with a double rocker is expedient. This significantly increases the adjusting range, more precisely the maximum expansion, while only having little additional complexity.

In order to actuate the drive means, an actuation connector is expediently provided for on a posterior end face. This arrangement on the end face affords the advantage of it being possible to reach the actuation connector through the same minimally invasive access, through which the intervertebral fusion implant according to the invention is also inserted. Hence, it is easily accessible, even in the case of a re-operation. The embodiment of the actuation connector as a screw coupling is particularly expedient. Here, this can, in particular, be a nut which is attached to the actuation spindle.

At least one through-hole is preferably formed in the cover surface and/or base surface. What this renders possible is that bone material can grow into the interior region of the implant in an improved manner. As a result of this, the sought-after fusion of the two adjacent vertebral bodies is accelerated.

### BRIEF DESCRIPTION OF THE DRAWINGS

The invention will be explained in more detail below on the basis of two exemplary embodiments, with reference being made to the attached drawing. In detail:

FIG. 1: shows a schematic view of an intervertebral fusion implant in accordance with a first exemplary embodiment, in the implanted state on vertebral bodies;

FIG. 2: shows a perspective view of the first exemplary embodiment, in an assembly position;

FIG. 3: shows a perspective view of the first exemplary embodiment, in the working position thereof, in the expanded state;

FIG. 4: shows a perspective view of a second exemplary embodiment, in an assembly position; and

FIG. 5: shows a perspective view of the second exemplary embodiment, in the working position thereof, in the expanded state.

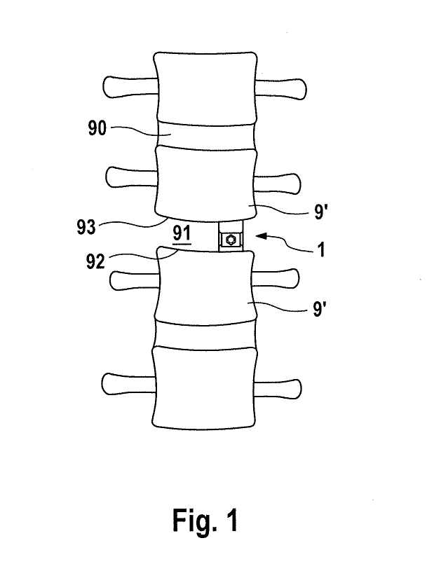

### DETAILED DESCRIPTION OF THE INVENTION

An intervertebral fusion implant, denoted by reference sign 1 in its entirety, is provided for implantation in an intervertebral space 91 between two immediately adjacent vertebral bodies 9, 9′. In a physiologically intact vertebral column, an intervertebral disk 90 is located in the intervertebral space between the vertebrae. This intervertebral disk may undergo degeneration as a result of disease or wear, with the result that it has to be at least partially resected. In order to achieve sufficient support of the intervertebral space 91, despite the loss of intervertebral disk material, and to thereby prevent collapse of the vertebral column, the intervertebral fusion implant 1 is inserted into the intervertebral space 91. It provides a supporting action and thus facilitates fusion of the two adjacent vertebrae 9, 9′ in a natural way through bone growth.

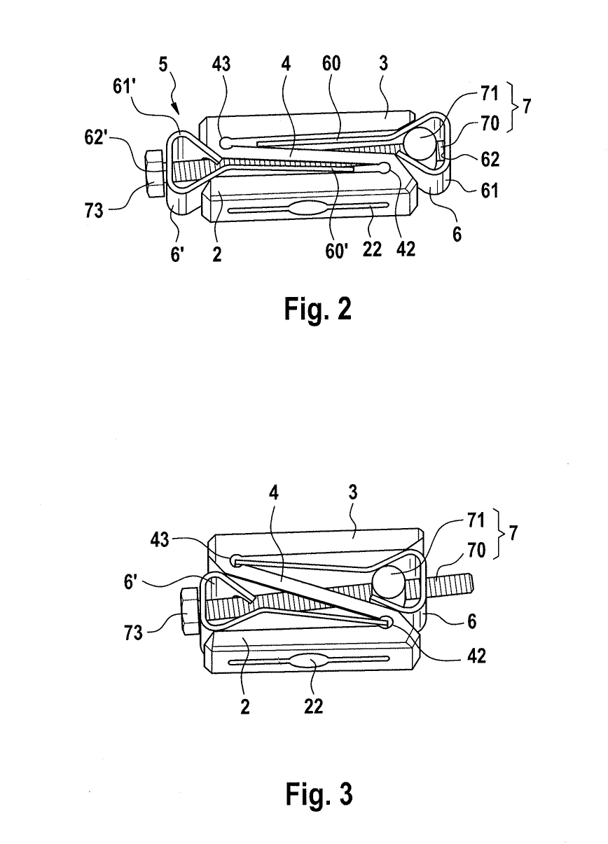

A first exemplary embodiment of an intervertebral fusion implant according to the invention, depicted in FIGS. 2 and 3 and denoted in its entirety by reference sign 1, comprises a base piece 2 and a cover piece 3 with a rocker 4 situated therebetween. Here, the rocker 4 is connected integrally to the base piece 2 and the cover piece 3 by two hinges 42, 43 at the outer ends thereof. The hinges and 43 are arranged at the opposite ends of the rocker 4 and also at the sides of the base piece 2 and of the cover piece 3 situated opposite to one another. In the depicted exemplary embodiment, the hinge 42 connecting the rocker 4 to the base piece 2 is situated at the anterior end (on the right-hand side in the illustration in FIGS. 2 and 3), while the hinge 43 connecting the rocker 4 to the cover piece 3 is situated at the posterior end. Overall, the result of this is a type of Z-shape for the first exemplary embodiment.

A perforation 22 is arranged in the base piece 2. It connects the space below the base piece 2 with the interspace between the base piece 2 and the rocker 4 or the cover piece 3.

A spreader clip 6 has been inserted into the interspace, open in the anterior direction, between the rocker 4 and the cover piece 3. It has a guide piece 60, which, in the manner of a tongue, slides into the interspace between rocker 4 and cover piece 3. In the assembly position, as depicted in FIG. 2, the free end thereof is at a distance from the hinge 43 between the cover piece 3 and the rocker 4. At its other end, the guide piece 60 continues integrally into a holding loop 61. The holding loop has a U-shape narrowing toward the top, wherein the one limb of the U bears on the cover piece 3 and the other limb of the U bears on the rocker 4 in the region of the hinge 42. The two limbs are connected by a base piece of the U-shaped loop 61, which forms the anterior end of the spreader clip 6. There, provision is made for an opening 62 for a screwing spindle 70 to pass therethrough. In this case the screwing spindle 70 extends through the rocker 4 from the posterior end, parallel to the guide piece 60, through the interior space of the U-shaped holding loop 61 and ends in the region of the opening 62 in the assembly position.

The size of the loop 61 is dimensioned such that a counterbearing 71 for the screwing spindle 70 is held therein. In the depicted exemplary embodiment, the counterbearing 71 is configured as a cylindrical body, which has a passage bore with a female thread complementary to the screwing spindle 70 across the cylinder axis thereof.

In the depicted exemplary embodiment, provision is made for a second spreader clip 6′ which, in a complementary manner, is inserted in the interspace, open in the posterior direction, between the base piece 2 and the rocker 4. It has a similar design with a guide piece 60′ and a U-shaped loop 61′ with an end-faced opening 62′. The other end of the screwing spindle 70 is pushed through this opening, with a screw coupling 73 in the form of a welded-on nut being situated at the posterior ends of the screwing spindle 70 situated outside of the U-shaped loop 61′.

The intervertebral fusion implant according to the invention is inserted at its intended location in the intervertebral space 91 in the depicted assembly position (see FIG. 2) through a minimally invasive access. After the insertion, it is expanded so as, with its base piece 2, to reach the upper cover plate 92 of the lower adjacent vertebra and so as, with its cover piece 3, to reach the lower cover plate 93 of the upper adjacent vertebra 9′.

Here, the intervertebral fusion implant is actuated for expansion purposes as follows: an appropriately fitting wrench is applied to the nut as a screw coupling connector 73. By rotating the nut with the screwing spindle 70 welded thereon, a contraction means formed by the screwing spindle 70 and the counterbearing 71 as well as the nut 73 contracts such that the spreader clip 6 is pulled into the interspace between rocker 4 and cover piece 3. A corresponding statement applies to the spreader clip 6′ arranged opposite thereto. By pulling the spreader clips 6, 6′ into the respective interspaces, the U-shaped loops 61, 61′ with their tapering limbs come to bear against the outer ends of the cover piece 3 and of the hinge 42 for the spreader clip 6 and the base piece 2 and the hinge 43 for the spreader clip 6′, as a result of which the respective interspace between the rocker and the base piece 2 or the cover piece 3 is expanded.

As a result, the implant expands in height, i.e. the distance between the base piece 2 and the cover piece 3 increases. Such an expansion state, as is provided for the retention of the implant, is depicted in FIG. 3. It is referred to as working position. It is possible to identify that the spreader clips 6, 6′ pulled inwardly by the drive means 7 expand the interspaces and hence result in an increased distance between the base piece 2 and the cover piece 3.

It should be noted that this expansion is continuous and can also be wholly or partly reversed at any time by turning back the screwing spindle 70. It should furthermore be noted that the granted arrangement of screwing spindle 70 with the counterbearing 71, which has a passage bore with a female thread, brings about self-retention. Hence, there can be no reduction in the distance without an explicit appropriate actuation by the surgeon.

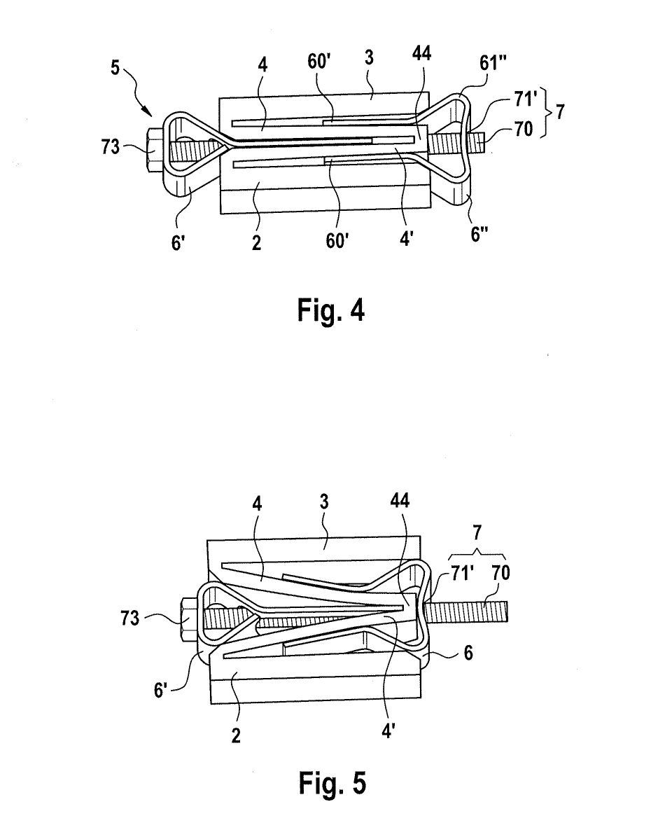

FIGS. 4 and 5 depict a second exemplary embodiment. Similar parts have been provided with the same reference sign as in the first exemplary embodiment.

The substantial difference from the first exemplary embodiment lies in the fact that the rocker is embodied as a double rocker 4, 4′ with an additional hinge 44 between the two rockers 4, 4′. As a result, the Z-shape of the first exemplary embodiment is extended to a type of M-shape. Hence, there are two interspaces on the anterior side, namely, firstly the ones between the cover piece 3 and the rocker 4, as in the first exemplary embodiment as well, and now also the one between the base piece 2 and the additional rocker 4′. The corresponding anterior spreader clip 6 is therefore modified in such a way that it now has two guide pieces 60, 60′, with the additional guide piece 60′ engaging in the newly created interspace, open in the anterior direction, between the base piece 2 and the additional rocker 4′.

A female thread 71′, which is arranged in an opening on the base part of the U-shaped loop 61″, acts as counterbearing. Otherwise, the functionality corresponds to that of the first exemplary embodiment. When actuating the drive means on the screw head 73, the two spreader clips 6″, 6′ are pulled into the interspaces, as a result of which the interspaces are spread and the cover piece 3 moves away from the base piece 2.

1. An intervertebral fusion implant for fusing two adjacent vertebrae, comprising a base piece, a cover piece, and a spreader clip, wherein the base piece and the cover piece are connected by a rocker situated between the base piece and the cover piece, and the spreader clip is situated on the rocker, and a drive device is provided for spreading the spreader clip.

2. The intervertebral fusion implant of claim 1, further comprising a second spreader clip situated opposite the first spreader clip.

3. The intervertebral fusion implant of claim 1, wherein the drive device comprises a contraction element.

4. The intervertebral fusion implant of claim 1, wherein a screwing spindle forms an actuator for the drive device.

5. The intervertebral fusion implant of claim 1, wherein the rocker has integral hinges.

6. The intervertebral fusion implant of claim 5, wherein the rocker is integral with one or both of the base piece and the cover piece.

7. The intervertebral fusion implant of claim 1, wherein the rocker comprises a double rocker.

8. The intervertebral fusion implant of claim 1, wherein an actuation connector for the drive device is arranged on a posterior end face.

9. The intervertebral fusion of claim 8, wherein the actuation connector comprises a screw coupling.

10. The intervertebral fusion implant of claim 1, wherein at least one through-hole is formed in one or both of the cover piece and the base piece.
